(19) United States
(12) Patent Application Publication
(10) Pub. No.: US 2010/0226920 A1
(43) Pub. Date: Sep. 9, 2010

(54) MEDICAL DELIVERY DEVICE FOR THERAPEUTIC PROTEINS BASED ON SINGLE DOMAIN ANTIBODIES

(75) Inventors: Gerrit Franciscus Landolt, Mariakerke (BE); Torsten Dreier, Sint-Martens-Latem (BE)

Correspondence Address: WOLF GREENFIELD & SACKS, P.C., 600 ATLANTIC AVENUE, BOSTON, MA, 02210-2206, US

(73) Assignee: ABLYNX N.V., Ghent-Zwijnaarde (BE)

(21) Appl. No.: 12/225,644

(22) PCT Filed: Mar. 26, 2007
(86) PCT No.: PCT/EP2007/002657

§ 371 Date: May 25, 2010

Related U.S. Application Data
(60) Provisional application No. 60/786,126, filed on Mar. 27, 2006.

(51) Int. Cl. A61K 39/395; A61P 35/00; A61P 29/00; A61P 25/28; A61M 5/178; G01N 33/53; C12Q 1/70

(52) U.S. Cl. 424/134.1; 424/135.1; 604/187; 436/501; 435/5; 435/7.1

(57) ABSTRACT

The present invention relates to a pen-style administration device for administering therapeutic or diagnostic therapeutic proteins that are based on single domain antibodies, and methods of using such a device in therapies or diagnoses.

### FIELD OF INVENTION

The present invention relates to a pen-style administration device for administering therapeutic or diagnostic therapeutic proteins that are based on single domain antibodies, and methods of using such a device in therapies or diagnoses.

### BACKGROUND OF THE INVENTION

Medicine injector pens have been developed to permit a patient to conveniently and accurately self-administer proper doses of medicine. Medicine pens are so named due to their general resemblance to the writing instrument in their elongated shape and overall length. While medicine pens have traditionally used for insulin administration, they will find use in any treatment regimen that requires frequent doses of drugs, that can not be delivered orally. The pen devices are best used with medications that are stable at room temperature or when stored in a fridge.

Polypeptide therapeutics and in particular antibody-based therapeutics have significant potential as drugs because they have exquisite specificity to their target and a low inherent toxicity. However, an antibody that has been developed for a therapeutic target through the standard monoclonal process (e.g., in mice) requires additional modifications to be used in humans. Because of their non-human sequences, unmodified traditional antibodies would induce an unwanted immunological reaction in a human individual upon administration. Several antibody-modification processes have been developed, including those methods that join parts of human and non-human antibody molecule to make a “chimeric” antibody, and those methods that modify specific amino acid residues to make modified antibodies (e.g., “humanization” and “veneering”) The latter methods are time consuming and involve an iterative process in which amino acids or groups of amino acids are changed to more closely resemble a human antibody to reduce antigenicity.

To circumvent the problems associated with traditional antibodies and the aforementioned modified antibodies, single domain antibodies have been developed.

For example, single domain antibodies which are referred to as “Domain antibodies” or “dAb's” (which are based on or derived from the heavy chain variable domain (V_{H}) or the light chain variable domain (V_{L}) of traditional 4 chain antibody molecules) were shown to be functional (see, e.g., Ward et al. 1989 Nature 341, 544-546), and subsequently had to be optimized for binding and solubility properties by phage display technology (Jespers et al 2004, J. Mol. Biol. 337, 893-903).

These problems were addressed in the development of a subsequent single domain antibody technology: Nanobodies®. Nanobodies® are based on the discovery that several species, including camelids (e.g., camels and llamas) and cartilaginous fish (e.g., sharks), have evolved high-affinity single V-like domains. Both the camelid single domain antibody (V_{HH}) and the shark version (V-NAR) are soluble, can be produced in vitro (Conrath et al. 2005, J. Mol. Biol. 350, 112-125), and show only a minimal immune response in humans. Also, Nanobodies® can be obtained by immunizing a Camelid with the desired antigen and then isolating the V_{HH} sequences (i.e., amino acid or nucleic acid) from B-cells obtained from the Camelid. Thus, compared to “dAb's”, Nanobodies® have the advantage of being derived from a process that involves in vivo maturation, which often leads to high affinity for the intended antigen (i.e. compared to techniques that involve the screening of large synthetic or naive libraries).

At present it is envisaged that single domain antibodies will be administered by a health care provider in a medical office or hospital setting. However, there are drawbacks to this administration scheme, particularly for patients that require repeated administration or rapid administration of the single domain antibodies, such as in treatment of chronic diseases or acute disorders requiring immediate intervention.

### SUMMARY OF THE INVENTION

To provide for improved administration of single domain antibodies, the present invention includes a pen-style administration device for administering therapeutic or diagnostic single domain antibodies. The invention also includes methods of using such a device in therapies or diagnoses.

According to one aspect of the invention, a pen-style administration device for the administration of a liquid medicine or diagnostic that includes a single domain antibody or a polypeptide construct with at least one single domain antibody is provided. The pen-style device includes a housing and a reservoir disposed in the housing constructed and arranged to hold a liquid medicine or diagnostic. The reservoir is in fluid connection with an outlet, and the outlet is constructed and arranged for administering the liquid medicine or diagnostic to a subject. In one preferred embodiment the outlet is an injection needle. In another embodiment the administration device has an actuator to dispense the liquid medicine or diagnostic from the reservoir. In some embodiments the administration device is adjustable for delivering variable doses of liquid medicine or diagnostic. In another preferred embodiment the administration device is a prefilled syringe for single use or multiple uses.

The liquid medicine or diagnostic administered with the administration device contains a single domain antibody or a polypeptide construct with at least one single domain antibody, preferably a Nanobody®. In preferred embodiments the single domain antibody is a V_{H}, V_{HH}, a camelized V_{H} or a humanized V_{HH}. In another preferred embodiment the to polypeptide construct contains at least two single domain antibodies, i.e. to provide a so-called multivalent (bivalent, trivalent, etc.) construct. In another embodiment the single domain antibodies in such a multivalent construct are connected by a chemical linker or a peptide linker.

In some embodiments a multivalent polypeptide construct comprises at least two single domain antibodies that bind to the same target. Such a multivalent construct may have a higher avidity for the target than a polypeptide that comprises only one (i.e., a “monovalent”) single domain antibody, in particular when the target is a multimer (such as TNF).

In some embodiments such a polypeptide construct comprises at least two single domain antibodies that bind to at least two different targets, i.e., to provide so-called “multispecific” (bispecific, trispecific, etc.) constructs. In yet another embodiment at least one of the targets is a serum protein. The serum protein may be serum albumin, serum immunoglobulin, thyroxine-binding protein, transferrin or fibrinogen. Binding of the single domain antibody to the serum protein preferably increases the half-life of the single domain antibody or polypeptide construct.

In other embodiments the single domain antibody or polypeptide construct includes a non-single domain antibody functional group or is coupled to a non-single domain antibody functional group through a linker. In some embodiments the linker is a chemical linker or a peptide linker. In other embodiments the non-single domain antibody functional group that is part of the polypeptide construct is a serum protein. The serum protein may be serum albumin, serum immunoglobulin, thyroxine-binding protein, transferrin or fibrinogen. Binding of the single domain antibody to the serum protein preferably increases the half-life of the single domain antibody or polypeptide construct.

In another aspect of the invention, methods for treating and/or preventing and/or alleviating a disease or disorder are provided. The methods include administering to a subject in need of such a treatment an effective amount of liquid medicine using a pen-style administration device. In some embodiments the disease or disorder is an inflammatory disorder, an autoimmune disease, a cancer, a neurodegenerative disorder or a genetic disorder. In other embodiments the single domain antibody binds to a target of foreign origin, a host derived cellular target or a host derived non-cellular target. The target of foreign origin may be a virus, a bacteria, a toxin, a radioactive compound or a drug.

In another aspect of the invention, methods for diagnosing a disease or disorder are provided. The methods include administering to a subject an effective amount of diagnostic using a pen-style administration device. In some embodiments the disease or disorder is an inflammatory disorder, an autoimmune disease, a cancer, a neurodegenerative disorder or a genetic disorder. In other embodiments the single domain antibody binds to a target of foreign origin, a host derived cellular target or a host derived non-cellular target.

These and other aspects and embodiments of the invention are described in greater detail below.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### DETAILED DESCRIPTION OF THE INVENTION

The invention provides a pen-style administration device for administering polypeptide therapeutics or diagnostics that are, or include, single domain antibodies, such as Nanobodies® (Ablynx N. V., Ghent, Belgium). The delivery of single domain antibodies by pen-style devices provides certain benefit, including ease of use, reduced risk of contamination and accurate dosing, over the other administration devices and methods. These benefits are, in some instances, unexpected in view of previously known administration of proteins (e.g., insulin) using pen-style devices.

### Pen-Style Device

Pen-style devices are well known in the art, including the devices used for self-administration of insulin by diabetics. Exemplary devices include the NovoPen® marketed by Novo Nordisk A/S (Bagsværd, Denmark), and the Humalog® prefilled pen device marketed by Eli Lilly and Company (Indianapolis, USA). A pen-styled device in certain embodiments includes a single domain antibody medicine or diagnostic filled reservoir connected with an outlet (e.g., a needle) through which medicine or diagnostic may be injected into a user. To inject a pre-determined quantity of the single domain antibody medicine or diagnostic, the pen is maneuvered such that the outlet tip (e.g. the needle) is inserted (e.g., intravenously, subcutaneously, intramuscularly, etc.) into the user. A button or knob that projects from the distal end of the device housing is depressed and moved relative to the device housing, which results in a plunger moving within the cartridge axially toward the injection needle to force medicine from the reservoir and out through the needle. This knob is typically depressed in line with and toward the needle by a finger, such as the thumb, of the hand in which the medicine pen is being held. In order to so drive the knob, an axial force must be applied to the medicine pen knob.

Thus, in one preferred embodiment the present invention provides a delivery apparatus including a housing elongated in an axial direction, a reservoir constructed and arranged to hold a liquid medicine or diagnostic, the reservoir disposed in said housing and in fluid connection with an outlet, an outlet in fluid communication with the container to receive the medication forced therefrom, and an actuator movable relative to the housing from a first position to a second position. The actuator can be part of the drive assembly that is adapted to interact with the medication container to deliver the single domain antibody or diagnostic from the reservoir and through the outlet upon movement of the actuator. In another embodiment, the drive assembly includes an actuator movable relative to the housing from a first position to a second position and is adapted to interact with the reservoir to deliver the single domain antibody medicine or diagnostic from the reservoir and through the outlet upon movement of the actuator from the first position to the second position. The pen-style device is operable to control a quantity of medication delivered from the container by the drive assembly (see, e.g., U.S. Pat. No. 6,454,746).

In another embodiment, the invention provides a pen-style administration device for repetitive injection of individually set or pre-determined doses of a single domain antibody medicine or diagnostic, from the reservoir. The pen-style administration device includes a housing, a reservoir containing a single domain antibody medicine or diagnostic to be expelled, an actuator adapted to expel a dose of medicine from the reservoir and a dose setting assembly mounted in or on the housing. Such doses may be pre-determined or may be determined by user adjustment of the device (e.g. U.S. Pat. No. 6,899,699). In another embodiment, a pen-style administration device includes a housing, reservoir and outlet as described above, and a rotatable dose setting device mounted on the housing and capable of being moved to a selected set position, a latch arranged to retain the setting device in the set position, and a release member that is constructed and arranged to release the latch to cause the set dose to be expelled (e.g. U.S. Pat. No. 5,104,380). In another embodiment, the invention relates to a single-use device for injection. The device comprises a pre-filled syringe, which is provided with a front nose, and supports an injection needle which is protected by a protective cap, and a syringe body which accommodates the said syringe. Injection of the medicine or diagnostic can be carried out by one thrust on the actuator (e.g. U.S. Pat. No. 6,585,702).

The delivery of a medicine can be done through transdermal injections or infusions, i.e. delivery of agents through the skin to a site beneath the skin. Transdermal injections and infusions include subcutaneous, intramuscular or intravenous routes of administration. In another embodiment the injection can be executed into the intradermal compartment of a subject's skin (e.g. U.S. published Application 2005/0180952). In another embodiment the transdermal delivery can be achieved through methods that do not involve a needle. In one embodiment the delivery system is a hypodermic injection system that allows for the generation of a high pressure liquid jet capable of passing through the skin (e.g., U.S. Pat. No. 6,258,063).

The liquid medicine or diagnostic can be any liquid medicine that contains at least one single domain antibody or Nanobody®, or at least one protein or polypeptide that comprises or essentially consists of at least one single domain antibody or Nanobody® (including but not limited to multivalent or multispecific constructs as described herein), and that can be administered to a subject to patient using the pen-style administration device described herein. For example, and without limitation, the liquid medicine or diagnostic can be a solution, suspension, emulsion or other pharmaceutically acceptable liquid formulation that comprises at least one such single domain antibody, Nanobody, protein or polypeptide. Preferably, the liquid medicine is a solution (for example an aqueous solution, which is generally preferred, although the invention in its broadest sense is not limited thereto). The solution or other liquid formulation may also contain one or more further additives for such solutions or formulations known per se, which will be clear to the skilled person, and for which reference is made for example made to the standard handbooks and to the prior art mentioned herein, as well as to the further disclosure herein.

### Single Domain Antibodies

The use of traditional antibodies derived from sources such as mouse, sheep, goat, rabbit etc., is challenging for a number of reasons as described above. In addition, traditional antibodies are not stable at room temperature, and have to be refrigerated during preparation and storage. Furthermore, the manufacture or small-scale production of said antibodies is expensive because the mammalian cellular systems necessary for the expression of intact and active antibodies require high levels of support in terms of time and equipment, and yields are very low. Another disadvantage is the large size of conventional antibodies, which would restrict tissue penetration, for example, at the site of a solid tumor. Furthermore, traditional antibodies have a binding activity which depends upon pH, and hence are unsuitable for use in environments outside the usual physiological pH range such as, for example, in treating colorectal cancer.

The problems of traditional antibodies have been addressed through the development of single domain antibodies, which consist of the smallest known antigen-binding fragments of antibodies, ranging from 11 kDa to 15 kDa. Single domain antibodies include Nanobodies® (Ablynx N. V., Ghent, Belgium), which are variable domains of heavy chain antibodies of Camelid species and also include heavy chain variable domains of other species in which “camelizing” alterations to amino acid sequence have been made. Another type of single domain antibodies are Domain Antibodies or “dAb's” (Domantis Inc., Waltham, Mass., USA), which contain the variable regions of the heavy and light chains of the human immunoglobulins (V_{H} and V_{L} respectively) and are optimized for binding and solubility properties. Both Nanobodies® and Domain Antibodies are currently being developed for therapeutic use.

### Nanobodies®

The variable domains present in naturally occurring heavy chain antibodies, as occur in the camelid species, will be referred to as “V_{HH} domains”, in order to distinguish them from the heavy chain variable domains that are present in conventional 4-chain antibodies (which will be referred to herein as “V_{H} domains”) and from the light chain variable domains that are present in conventional 4-chain antibodies (which will be referred to herein as “V_{L} domains”). As will become clear from the further discussion below, V_{HH} domains have a number of unique structural characteristics and functional properties, which make isolated V_{HH} domains (as well as Nanobodies®, which share said structural characteristics and functional properties with the naturally occurring V_{HH} domains) highly advantageous for use as functional antigen-binding domains or proteins (i.e. compared to isolated naturally occurring V_{H} domains or V_{L} domains, which by themselves are not suitable as antigen-binding units, for the reasons discussed herein).

Isolated V_{HH} domains—which have been “designed” by nature to functionally bind to an antigen without the presence of, and without any interaction with, a light chain variable domain—can be used as such as a single, relatively small, functional antigen-binding structural unit, domain or protein. This also distinguishes the V_{HH} domains from the V_{H} and V_{L} domains of conventional 4-chain antibodies, which by themselves are generally not suited as antigen-binding proteins or domains, but need to be combined in some form or another to provide a functional antigen-binding unit, as in for example conventional antibody fragments or in scFv's (which consist of a V_{H} domain covalently linked to a V_{L} domain).

Because of these unique properties, the use of V_{HH} domains and Nanobodies® as antigen-binding proteins or antigen-binding domains (i.e. as part of a larger protein or polypeptide) offers significant advantages over the use of conventional V_{H} and V_{L} domains, scFv's or conventional antibody fragments (such as Fab- or F(ab)₂-fragments):
only a single domain is required to bind an antigen with high affinity and with high selectivity, so that there is no need to have two separate domains present, nor to assure that these two domains are present in the right spatial conformation and configuration (i.e. through the use of especially designed linkers, as with scFv's);
V_{HH} domains and Nanobodies® can be expressed from a single gene and require no post-translational folding or modifications;
V_{HH} domains and Nanobodies® can easily be engineered into multivalent and multispecific formats (as further discussed below);
V_{HH} domains and Nanobodies® are highly soluble and do not have a tendency to aggregate (as with the mouse-derived antigen-binding domains described by Ward et al., Nature, Vol. 341, 1989, p. 544);
V_{HH} domains and Nanobodies® are highly stable to heat, pH, proteases and other denaturing agents or conditions;
V_{HH} domains and Nanobodies® are easy and relatively cheap to prepare, even on a scale required for production. For example, V_{HH} domains, Nanobodies® and proteins/polypeptides containing the same can be produced using microbial fermentation, and do not require the use of mammalian expression systems, as with for example conventional antibody fragments;
V_{HH} domains and Nanobodies® are relatively small compared to conventional 4-chain antibodies and antigen-binding fragments thereof, and therefore show high(er) penetration into tissues (including but not limited to solid tumors) than such conventional 4-chain antibodies and antigen-binding fragments thereof;
V_{HH} domains and Nanobodies® can show so-called cavity-binding properties, and can therefore also access targets and epitopes not accessible to conventional 4-chain antibodies and antigen-binding fragments thereof. For example, it has been shown that V_{HH} domains and Nanobodies® can inhibit enzymes (see for example WO 97/49805; Transue et al., Proteins: structure, function, genetics, 32: 515-522 (1998); Lauwereys et al., EMBO J., Vol. 17, No. 13, p. 3512-3520).

Naturally occurring V_{HH} domains can be used as Nanobodies®. In addition, as also described below, the amino acid sequences of naturally occurring V_{HH} domains, and/or the nucleic acids and/or nucleotide sequences encoding the same, can be used as a starting point for developing, designing and/or preparing Nanobodies®, e.g. by using one of the various methods known to one skilled in the art.

One particularly preferred, but non-limiting class of Nanobodies® are Nanobodies® of which the amino acid sequence, compared to the sequence of a naturally occurring V_{HH} domain, has been “humanized”, i.e. by replacing one or more of the amino acid residues in the amino acid sequence of a naturally occurring V_{HH} domain with the amino acid residue(s) that occur at the corresponding position(s) of a conventional human V_{H} domain.

Another non-limiting class of Nanobodies® are Nanobodies® of which the amino acid sequence, compared to the sequence of a naturally occurring V_{H} domain, and in particular compared to the sequence of a naturally occurring V_{H} domain from a human being, has been “camelized”, i.e. by replacing one or more of the amino acid residues in the amino acid sequence of a naturally occurring V_{H} domain with one of the “hallmark residues” of a camelid antibody (see for example also WO 94/04678). As further described herein, the invention also generally comprises Nanobodies® comprising one or more of such camelizing substitutions, irrespective of the way these Nanobodies® have been generated or obtained (for example, by camelization, by synthesis de novo or in any other way).

It should also be noted that Nanobodies can for example also be obtained by “camelizing” a naturally occurring V_{H} domain from another species of mammal (i.e. a V_{H} domain from a naturally occurring conventional 4-chain antibody) such as from a human being, i.e. by replacing one or more amino acid residues in the amino acid sequence of said V_{H} domain by one or more of the amino acid residues from the camelid antibody. This can be performed in a manner known per se, which will be clear to the skilled person, for example as described in WO 94/04678 or as further known in the art. Such camelization may preferentially occur at amino acid positions which are present at the V_{H}-V_{L} interface and at the so-called Camelidae hallmark residues (see for example also WO 94/04678).

Exemplary patents and applications describing various aspects of Nanobodies® include: U.S. Pat. Nos. 5,759,808, 5,800,988, 5,840,526, 5,874,541, 6,005,079, 6,015,695, 6,765,087, and 6,838,254; US published patent applications 2003/0088074, 2004/0248201, 2004/0253638, 2005/0214857, 2005/0037358, 2005/0048060, 2005/0054001, 2005/0130266 and 2006/0034845; and PCT published applications WO 97/49805, WO 03/035694, WO 03/054016, WO 03/055527, WO 2004/062551, WO 2004/041867, WO 2004/041865, WO 2004/041863, WO 2004/041862, WO 2004/041867, WO 2005/044858, WO 2006/040153, WO 2006/122786 and WO 2006/122825.

In another embodiment, Nanobodies® are identified by Nanoclone™, a screening process comprising the direct sorting of single antigen-specific B-cells from immunized animals such as llamas. Reference may be made to patent applications of Ablynx entitled “Method For Generating Variable Domain Sequences Of Heavy Chain Antibodies”.

### Domain Antibodies

Human single domain antibodies have also been developed. The domains have been designed to have minimal hydrophobic elements, thereby minimizing the change of aggregation (Jespers et al. 2004, J. Miol. Biol. 893-903). These antibodies are being developed for therapeutics under the name Domain Antibodies (Domantis Inc., Waltham, Mass., USA), the characteristics of which are described below. The single domains of the antibodies are based on the human V_{H} fragment. However, since the human V_{H} fragment by itself has solubility problems and does not possess the strong binding properties of its parent molecule, the sequences of the domain have been optimized using phage display technology. Analysis of the structures and sequences of antibodies has shown that five of the six antigen binding loops (H1, H2, L1, L2, L3) possess a limited number of main-chain conformations or canonical structures (Chothia and Lesk (1987) J. Mol. Biol., 196: 901; Chothia et al. (1989) Nature, 342: 877) allowing for prediction of the loop lengths and key residues of the main-chain conformations of H1, H2, L1, L2 and L3 encoded by the majority of human antibody sequences (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (clue to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1). Analyzing the single domain antibodies in detail has allowed for focused efforts of ‘optimizing’ the antibody, balancing the need to introduce non-hydrophobic residues, but also minimizing the immune response in human subjects. Exemplary patents and applications describing various aspects of Domain Antibodies include: US published patent applications 2004/0058400, 2004/0110941, 2004/0127688, 2004/0192897, 2004/0219643, 2006/0063921 and 2006/0002935; and PCT published patent applications WO 03/002609, WO 2004/003019, WO 2004/058821, WO 2004/058822, and WO 2005/118642).

Phage-display technology also can be used for the in vitro selection of human and camelid single domain antibodies against a wide range of target antigens. Synthetic libraries have been used to overcome the inherent biases of the natural repertoire which can limit the effective size of phage libraries constructed from rearranged V genes. Human antibody frameworks can be pre-optimized by synthesizing a set of genes that have consensus framework sequences and incorporate amino acid substitutions shown to improve folding and expression. However, it is desirable to use artificial human antibodies which will not be recognized as foreign by the human immune system, the use of consensus frameworks preferably resembles human sequences.

### Multivalent and Multispecific Polypeptide Constructs

In order to further improve the avidity (i.e. for a desired antigen) of polypeptides that comprise single domain antibodies, and/or to provide constructs that can bind to two or more different antigens, two or more single domain fragments can be combined in a single polypeptide construct, resulting in a multivalent and/or multispecific polypeptide construct. The antibody domains can be coupled to each other directly (e.g. as a fusion protein) or using polypeptide or non-polypeptide linkers. In addition to increasing the binding to the antigen, multiple binding to a target can also increase the therapeutic activity of the multivalent polypeptide relative to the individual single domain antibodies. Multivalency does not have to be limited to two single domain antibodies, and as such, multivalent polypeptide constructs can be trimers and tetramers etc. of the same or different single domain antibodies. In some embodiments single domain antibodies that bind to different targets are coupled to each other resulting in multispecific polypeptide constructs. In some embodiments one of the single domain antibodies of the multispecific polypeptide constructs binds a serum protein, thereby increasing the half-life of the polypeptide construct. In some embodiments the serum protein is serum albumin, serum immunoglobulin, thyroxine-binding protein, transferrin or fibrinogen. In some embodiments the multispecific polypeptide constructs also can comprise two or more single domain antibodies that bind to the same target, thereby increasing the affinity for binding to a single antigen.

Single domain antibodies (or multivalent and/or multispecific polypeptide constructs) can also be coupled to polypeptides other than antibodies. Coupling of the single domain antibodies to non-antibody polypeptides can provide the single domain antibodies with an extra functionality and/or can increase their half-life. Individual single domain antibodies are small (˜15 kDa) and can be disposed of in the body through the kidneys. While single domain antibodies are more stable than traditional antibodies, their disposal through the kidneys may diminish their therapeutic effectiveness. Filtration by the kidneys can be prevented or reduced by increasing the size of individual single domain antibodies through multimerization as described above or through coupling to a larger protein, preferably a stable protein found in the bloodstream, like albumin. Coupling single domain antibodies to larger serum proteins will increase the half-life of the single domain antibodies. In some embodiments the serum protein is serum albumin, serum immunoglobulin, thyroxine-binding protein, transferrin or fibrinogen. In another embodiment single domain antibodies are coupled to a polypeptide that would give them additional functionalities. Examples include, but are not limited to, signaling peptides, binding peptides, peptide receptor ligands and functional enzymes.

Single domain antibodies (or multivalent and/or multispecific polypeptide constructs) can also be coupled to a non-polypeptide group. In one embodiment, the non-polypeptide group is a toxic agent. In another embodiment, the non-polypeptide group is a tracer. The non-polypeptide groups can be coupled to the single domain antibody through a linker as is described below.

Coupling the single domain antibody to a toxic agent will allow for delivery of the toxin to the antigen. This methodology can be used to introduce a toxic agent to a site where it is most effective (e.g. inside a tumor cell, or on the membrane of a tumor cell). The methodology can also be used to rid the bloodstream of unwanted products. The single domain antibody can bind to an unwanted antigen, which can be inactivated by the toxic agent that is attached to the single domain antibody. In contrast, if the toxic agent were not attached to a single domain antibody, it would not get in close proximity to its target product, or would not stay in sufficiently close proximity long enough, to inactivate the target. Single domain antibodies can also be coupled to tracers. This will allow for the monitoring of a specific target in the body. For instance, a single domain antibody that binds to a tumor antigen can be coupled to a radioactive tracer. The amount of radioactivity retained in the body and the localization of the tracer will help diagnose the amount of tumor cells in the body and can help determine the progress of a specific treatment regimen (See also below).

Amino acid linkers for use in multivalent and multispecific polypeptides will be clear to the skilled person, and for example include Gly-Ser linkers, for example of the type (GlyₓSer_{y})_{z}, such as for example (Gly₄Ser)₃ or (Gly₃Ser₂)₃, as described in WO 99/42077, hinge-like regions such as the hinge regions of naturally occurring heavy chain antibodies or similar sequences. Linkers can also provide some functionality for the multivalent or multispecific polypeptides. For example, linkers containing one or more charged amino acid residues can provide improved hydrophilic properties, whereas linkers that form or contain small epitopes or tags can be used for the purposes of detection, identification and/or purification.

Single domain antibodies can be connected to each other, to other polypeptides or to non-polypeptides groups through a number of non-peptide linkers. In one embodiment, the linker comprises an amido linker moiety, an amino linker moiety, a carbonyl linkermoiety, a carbamate linker moiety, a urea linker moiety, an ether linker moiety, a disulphide linker moiety, a succinamidyl linker moiety, a succinyl linker moiety, and combinations thereof. In other embodiments, the linker moiety is an ester including: carbonate (——OC(O)O——), succinoyl, phosphate esters (——O——(O)POH——O——), sulfonate esters, and combinations thereof. In another embodiment, the linker contains polyethylene glycol (PEG) with an average molecular weight of about 550 to about 10,000 daltons and is optionally substituted by alkyl, alkoxy, acyl or aryl.

Generally, for pharmaceutical use, the single domain antibodies and the polypeptide constructs of the invention may be formulated as a pharmaceutical preparation comprising at least one polypeptide of the invention and at least one pharmaceutically acceptable carrier, diluent or excipient and/or adjuvant, and optionally one or more further pharmaceutically active polypeptides and/or compounds. Suitable formulations are known in the art. For use in the administration devices of the invention, the formulation is a liquid or solution formulation suitable for transdermal administration (such as by intravenous, intramuscular or subcutaneous injections).

An effective amount of liquid medicine is a dosage of the single domain antibody or a polypeptide construct sufficient to provide a medically desirable result. The effective amount will vary with the particular condition being treated, the age and physical condition of the subject being treated, the severity of the condition, the duration of the treatment, the nature of the concurrent therapy (if any), the specific route of administration and like factors within the knowledge and expertise of the health practitioner. For example, an effective amount for treating cancer would be an amount sufficient to lessen or inhibit altogether cancer cell proliferation so as to slow or halt the development of or the progression of a tumor. In some embodiments the disease is cancer etc. An effective amount of a diagnostic is a dosage of the single domain antibody or a polypeptide construct sufficient to provide a medically relevant diagnosis. The effective amount will vary with the particular condition being diagnosed, the age and physical condition of the subject being diagnosed, the severity of the condition, the specific route of administration and like factors within the knowledge and expertise of the health practitioner.

In one embodiment, pen devices are used to administer antibodies used for diagnostics. Coupling a tracer to an antibody will allow for the determination of an amount of a specific antigen in vivo or in vitro. The tracer can be, but is not limited to an agent of fluorescent or radioactive origin. The diagnostic administered with the pen device can be used to determine the amount and location of a variety of antigens, for instance a solid tumor cell marker or peptide marker in the bloodstream. In most diagnostic assays, the diagnostics needs to be administered a set time prior to the assay. While the readout of the diagnostic assay will likely need to be performed by a health care official, being able to self administer the diagnostic dose will allow for one less trip to the clinic and savings in time and cost. Traditional antibodies can be used for the diagnostic assay in principle. However, single domain antibodies will be much preferred because of their stability and small size. They could for instance enter a tumor cell, allowing for a more complete diagnostic picture.

A variety of diseases can be treated using single domain antibodies as delivered by the pen-style administration device of the invention. Exemplary diseases include inflammatory o disorders, cancers, autoimmune diseases, neurodegenerative disorders, genetic disorders,

“Inflammatory disorders” include diseases such as rheumatoid arthritis, Crohn's disease, mastocytosis, asthmas, multiple sclerosis, inflammatory bowel syndrome and allergic rhinitis (see, e.g., US published application 2006/0058340). For example, in one embodiment, the pen-style administration device is used to administer (polypeptides comprising one or more) single domain antibodies against TNF-alpha, against IL-6 against IL-6R or against another protein or target involved in the IL-6 pathway (see for example the non-prepublished US provisional application US 60/873,012 by Ablynx N. V.), as for example described in WO 2004/041862, US 2005/0054001, US 2006/0034845, US 60/682332, WO 03/050531, WO 03/054016, US 60/782243,US 60/782246 and WO 06/122786, and in WO 04/003019 and WO 03/002609.

For the prevention and treatment of aggregation-mediated disorders and/or thrombotic disorders, for example polypeptides comprising one or more) single domain antibodies against vWF (as for example described in WO 2004/062551, US 10/541708 and US 60/683474 and WO 06/122825) may be formulated as and administered using the pen-like administration devices disclosed herein.

The cancer may be a carcinoma or a sarcoma but it is not so limited. For example, the cancer may be basal cell carcinoma, biliary tract cancer, bladder cancer, bone cancer, brain cancer, breast cancer, cervical cancer, choriocarcinoma, CNS cancer, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, acute lymphoid leukemia, acute myeloid leukemia, chronic lymphoid leukemia, chronic myeloid leukemia, cutaneous T-cell leukemia, hairy cell leukemia, liver cancer, non-small cell lung cancer, small cell lung cancer, lymphoma, follicular lymphoma, Hodgkin's lymphoma, Non-Hodgkin's lymphoma, melanoma, myeloma, multiple myeloma, neuroblastoma, oral cavity cancer, ovarian cancer, pancreatic cancer, prostate cancer, rectal cancer, renal cancer, cancer of the respiratory system, retinoblastoma, rhabdomyosarcoma, skin cancer, squamous cell carcinoma, stomach cancer, testicular cancer, thyroid cancer, cancer of the urinary system and uterine cancer (US published application 2006/0019923).

An “immune disorder” includes adult respiratory distress syndrome, arteriosclerosis, asthma, atherosclerosis, cholecystitis, cirrhosis, Crohn's disease, diabetes mellitus, emphysema, hypereosinophilia, inflammation, irritable bowel syndrome, multiple sclerosis, myasthenia gravis, myocardial or pericardial inflammation, osteoarthritis, osteoporosis, pancreatitis, rheumatoid arthritis, scleroderma, and ulcerative colitis (see, e.g., US published application 2003/0175754).

“Neurodegenerative disorder” or “neurodegenerative disease” or “neuropathology” refers to a wide range of diseases and/or disorders of the central and peripheral nervous system, such as Parkinson's disease, Alzheimer's disease (AD), amyotrophic lateral sclerosis (ALS), denervation atrophy, otosclerosis, stroke, dementia, multiple sclerosis, Huntington's disease, encephalopathy associated with acquired immunodeficiency disease (AIDS), and other diseases associated with neuronal cell toxicity and cell death (see, e.g., US published application 2006/0025337). For example, for the prevention and/or treatment of Alzheimer's disease, polypeptides comprising one or more) single domain antibodies against amyloid-beta (as for example described in US 60/718617 and WO 06/040153) may be formulated as and administered using the pen-like administration devices disclosed herein.

“Genetic disorders” include Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyly, breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset to diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, SRY: sex determination, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma pigmentosum or Zellweger syndrome. (see, e.g., 2005/0281781)

In addition, molecules that can be targeted (“targets”) by the single domain antibodies delivered by the pen-style administration device of the invention include, for example, targets of foreign origin, host derived cellular targets, and host derived non-cellular targets. Exemplary targets are listed below.

Foreign target agents include drugs, especially drugs subject to abuse such as heroin and other opiates, PCP, barbiturates, cocaine and derivatives thereof, benzodiazepines, etc., poisons, toxins such as heavy metals like mercury and lead, chemotherapeutic agents, paracetamol, digoxin, free radicals, arsenic, bacterial toxins such as LPS and other gram negative toxins, Staphylococcus Toxins, Toxin A, Tetanus toxins, Diphtheria toxin and Pertussis toxins, plant and marine toxins, virulence factors, such as aerobactins, radioactive compounds or pathogenic microbes or fragments thereof, including infectious viruses, such as hepatitis B, A, C, E and delta, CMV, HSV (type 1, 2 & 6), EBV, varicella zoster virus (VZV), HIV-1, -2 and other retroviruses, adenovirus, rotavirus, influenzae, rhinovirus, parvovirus, rubella, measles, polio, reovirus, orthomixovirus, paramyxovirus, papovavirus, poxvirus and picornavirus, prions, protists such as plasmodia tissue factor, toxoplasma, filaria; kala-azar, bilharziose, entamoeba histolitica and giardia, and bacteria, particularly gram-negative bacteria responsible for sepsis and nosocomial infections such as E. coli, Acynetobacter, Pseudomonas, Proteus and Klebsiella, but also gram positive bacteria such as staphylococcus, streptococcus, etc. Meningococcus and Mycobacteria, Chlamydiae, Legionnella and Anaerobes, fungi such as Candida, Pneumocystis carini, and Aspergillus, and Mycoplasma such as Hominis and Ureaplasma urealyticum (see, e.g., U.S. Pat. No. 5,843,440).

Host derived cellular and non-cellular targets against to which the single domain antibodies and constructs that are administered according to the invention may be directed will be clear to the skilled person and for example include, but are not limited to, all targets for which Nanobodies, dAb's or other single domain antibodies (or polypeptides comprising the same) have been proposed in the art (such as TNF-alpha, Von Willebrand factor, interleukins such as IL-6, amyloid-beta, etc., as well as the other targets mentioned in the prior art referred to herein), as well as more generally cellular and non-cellular targets for which antibodies or antibody fragments (including but not-limited to ScFv constructs) have been proposed in the art.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference, in particular for the teaching that is referenced hereinabove.

We claim:

1. A pen-style administration device comprising a housing, a reservoir constructed and arranged to hold a liquid medicine or diagnostic, the reservoir disposed in said housing and in fluid connection with an outlet, the outlet constructed and arranged for administering a liquid medicine or a diagnostic to a subject, wherein the liquid medicine or the diagnostic comprises a single domain antibody or a polypeptide construct containing at least one single domain antibody.

2. The administration device of claim 1, wherein the outlet comprises an injection needle.

3. The administration device of claim 1, wherein the administration device comprises an actuator for dispensing the liquid medicine or diagnostic from the reservoir.

4. The administration device of claim 1, wherein the administration device is adjustable for delivering variable doses of liquid medicine or diagnostic.

5. The administration device of claim 1, wherein the administration device is a pre-filled syringe for single use or multiple uses.

6. The administration device of claim 1, wherein the polypeptide construct comprises at least two single domain antibodies.

7. The administration device of claim 6, wherein the single domain antibodies are connected by a chemical linker or a peptide linker.

8. The administration device of claim 6, wherein the single domain antibodies bind to at least two different targets.

9. The administration device of claim 8, wherein at least one of the targets is a serum protein.

10. The administration device of claim 9, wherein the serum protein is serum albumin, serum immunoglobulin, thyroxine-binding protein, transferrin or fibrinogen.

11. The administration device of claim 9, wherein the binding of the single domain antibodies to the serum protein increases the half-life of the polypeptide construct.

12. The administration device of claim 1, wherein the polypeptide construct comprises a non-single domain antibody functional group or is connected to a non-single domain antibody functional group by a linker.

13. The administration device of claim 12, wherein the linker is a chemical linker or a peptide linker.

14. The administration device of claim 12, wherein the non-single domain antibody functional group is a serum protein.

15. The administration device of claim 14, wherein the serum protein is serum albumin, serum immunoglobulin, thyroxine-binding protein, transferrin or fibrinogen.

16. The administration device of claim 14, wherein the serum protein increases the half-life of the polypeptide construct.

17. The administration device of claim 1, wherein the single domain antibody is a V_{H} or V_{HH}.

18. The administration device of claim 1, wherein the single domain antibody is a camelized V_{H}.

19. The administration device of claim 1, wherein the single domain antibody is a humanized V_{HH}.

20. A method for treating and/or preventing and/or alleviating a disorder or disease state comprising administering to a subject in need of such a treatment an effective amount of a liquid medicine using the administration device of claim 1.

21. A method for diagnosing a disorder or a disease state comprising administering to a subject an effective amount of a diagnostic by using the administration device of claim 1.

22. The method of claim 20, wherein the disorder is an inflammatory disorder, an autoimmune disease, a cancer, a neurodegenerative disorder or a genetic disorder.

23. The method of claim 20, wherein the single domain antibody binds to a target of foreign origin.

24. The method of claim 23, wherein the target of foreign origin is a virus, a bacteria, a toxin, a radioactive compound or a drug.

25. The method of claim 20, wherein the single domain antibody binds to a host derived cellular target.

26. The method of claim 20, wherein the single domain antibody binds to a host derived non-cellular target.

27. The method of claim 21, wherein the disorder is an inflammatory disorder, an autoimmune disease, a cancer, a neurodegenerative disorder or a genetic disorder.

28. The method of claim 21, wherein the single domain antibody binds to a target of foreign origin.

29. The method of claim 28, wherein the target of foreign origin is a virus, a bacteria, a toxin, a radioactive compound or a drug.

30. The method of claim 21, wherein the single domain antibody binds to a host derived cellular target.

31. The method of claim 21, wherein the single domain antibody binds to a host derived non-cellular target.
